(19) United States
(12) Patent Application Publication
(10) Pub. No.: US 2022/0219007 A1
(43) Pub. Date: Jul. 14, 2022

(54) TREATMENT OF FUNGAL INFECTIONS

(71) Applicant: Emblation Limited, Alloa (GB)

(72) Inventors: Gary Beale, Alloa (GB); Eamon McErlean, Alloa (GB); Matthew Kidd, Alloa (GB); Shailesh Joshi, Alloa (GB)

(21) Appl. No.: 17/557,602

(22) Filed: Dec. 21, 2021

Related U.S. Application Data
(60) Provisional application No. 63/128,994, filed on Dec. 22, 2020.

(51) Int. Cl. A61N 5/02; A61P 31/10

(52) U.S. Cl. CPC A61N 5/022; A61P 31/10

(57) ABSTRACT

The present disclosure provides compositions, medicaments and methods for treating or preventing disorders, conditions and/or diseases affecting keratinised structures or tissues such as, for example, nails.

### RELATED APPLICATIONS

This application claims the benefit of the filing date under 35 U.S.C. 119(e) to U.S. provisional patent application No. 63/128,994, filed Dec. 22, 2020, the entire contents of which are expressly incorporated herein by reference in their entirety.

### FIELD OF THE INVENTION

The present disclosure provides compositions, medicaments and methods for treating or preventing disorders, conditions and/or diseases affecting keratinised structures or tissues such as, for example, nails.

### BACKGROUND OF THE INVENTION

Keratinised tissues and/or structures, in particular nails (fingernails and toenails), are prone to a range of diseases, conditions and/or disorders.

A Fungal nail infection (onychomycosis) also called (Tinea unguium) is a common problem that occurs when a fungal pathogen gains access to nail bed or nail matrix/nail plate. The global prevalence of dermatomycoses is as high as 20% according to the World Health Organization (WHO) and approximately 10% of the human population suffers from onychomycosis. This invasive condition results in destruction of nail keratin causing discolouration, thickening, separation and damage of the nail structure. Onychomycosis often occur as a result of physical trauma to the nail, from impacts/crushing to overzealous trimming of the nail. Maintaining high levels of foot moisture creates an optimal environment for fungal pathogens to thrive in the feet. In particular dermatophytes such as Trichophyton rubrum, T. mentagrophytes and Epidermophyton floccosum can exist causing athlete's foot (Tinea pedis). Trichophyton rubrum is the most common dermatophyte involved in onychomycosis which may establish from an athlete's foot infection however other dermatophytes can be involved. Proximal subungual onychomycosis is a fungal infestation of the recently formed nail plate which often occurs in immunocompromised individuals.

This highlights an aspect to the condition that exploits the immune response made more prevalent in these immunocompromised populations. This has been supported by research such as “The dermatophytes”, Weitzman I, Summerbell R C (1995) Clin Microbiol Rev 8: 240-259. and “The Immunologic Response to Trichophyton Rubrum in Lower Extremity Fungal Infections” Blutfield et al., (2015)—Journal of Fungi.

A successful treatment for chronic T. rubrum infection may involve targeting those mechanisms of T. rubrum that diminish the immune response and restore the inflammation mechanisms that the dermatophyte suppresses.

The ability of a fungal pathogenic yeast to manipulate the local immune environment has been discussed in “Cell wall mannoprotein of Candida albicans polarizes macrophages and affects proliferation and apoptosis through activation of the Akt signal pathway,” Jiang et al., International Immunopharmacology, vol. 72, pp. 308-321, 2019. T. rubrum is believed to manipulate the innate immune functions of keratinocytes via the activation of signalling pathways that regulate proliferation and differentiation in keratinocytes such as p38-MAPK “Trichophyton rubrum manipulates the innate immune functions of human keratinocytes”. Garcia-Madrid L A, Huizar-Lopez M D, Flores-Romo L, Islas-Rodriguez A E. Centr Eur J Biol. (2011) 6:902-10.10.2478/s11535-011-0060-6.

The most common therapy for onychomycosis is treatment with antifungal medications such as Terbinafine, Itraconazole or Fluconazole either topically or via oral ingestion. These medications often require prolonged usage (at least 1 year) and can report many side effects and adverse reactions. Other treatments include the use of lasers to directly heat and destroy the fungus which has not delivered a reliable option as highlighted in the literature “Laser Therapy for Onychomycosis: Fact or Fiction?”. Journal of Fungi. 1: 44-54. 2015. doi:10.3390/jof1010044, and Bristow, I R (2014). “The effectiveness of lasers in the treatment of onychomycosis: a systematic review”. Journal of foot and ankle research. 7: 34. doi:10.1186/1757-1146-7-34. PMC 4124774. PMID 25104974.

U.S. Pat. No. 7,292,893 provides methods for the treatment of keratinized tissue infected with a pathogen. Specifically, electromagnetic energy, for example microwave energy, is used to directly destroy or deactivate the pathogen thereby reducing or eliminating the pathogen from the keratinized tissue. Because of the severity of the treatment disclosed in this document, the user needs to be careful to limit exposure of the derma of the nail bed to the microwave energy. This prevents the technique from being used to affect the any of the wider effects of the pathogen on the host and its immune system.

An improved treatment method would be one where energy can be utilized to reverse the immune modulation advantage a fungal pathogen maintains. This immune modulation reversal could be employed to restore the local innate immune reaction to the pathogen, thus reversing the advantage the infection exploits in immunocompromised individuals and to a lesser extent in immunocompetent individuals.

### SUMMARY OF THE INVENTION

Disclosed herein are methods which use electromagnetic energy for the treatment and/or prevention of fungal infections in human and/or animal subjects.

In particular, the disclosed methods find application in the treatment and/or prevention of fungal infections in keratinised tissues and/structures. For example, the methods of this disclosure may be used to treat fungal nail infections or fungal infections of the unguis. Such infections may result in diseases and/or conditions which affect one or more of the structures and/or tissues of the nail including, for example, the derma, the matrix (matrix unguis); the nail bed; the nail plate (corpus unguis); the lunula, the nail sinus (sinus unguis); the nail root (Radix unguis); the epidermis; the dermis; the hyponychium; the epithelium; the cuticle; and the perionychium. The methods of this disclosure may be used to treat or prevent diseases and/or conditions affecting any of these tissues and/or structures.

The methods disclosed herein may be used to treat fungal infections in human and/or animal subjects. As such, the methods of this disclosure may not only find application in the treatment of infections, diseases and/or conditions of human nails (including the human fingernails and/or toenails), but also infections, diseases and/or conditions occurring in other keratinised tissues and/or structures such as, for example, hair, claws, hooves, talons, horns and the like.

A fungal infection of the nail may be referred to as onychomycosis or Tinea unguium.

Accordingly, the disclosure provides methods for the treatment or prevention of:
fungal nail infections;
fungal infections of the unguis;
onychomycosis; or
Tinea unguium.

A number of different fungal pathogens can cause onychomycosis including those referred to as dermatophytes and Fusarium. These terms embrace the genera Microsporum, Epidermophyton, Michrodochium and Trichophyton. The term also embraces the following species: Trichophyton rubrum, Trichophyton mentagrophytes and Epidermophyton floccosum.

Accordingly, the methods described herein may be for the treatment or prevention of diseases, conditions and/or infections caused or contributed to by any one or more of the below listed fungal pathogens:
(i) a dermatophyte;
(ii) a Fusarium;
(iii) a Microsporum sp.;
(iv) a Michrodochium sp.;
(v) a Epidermophyton sp.;
(vi) a Trichophyton sp.;
(vii) Trichophyton rubrum;
(viii) Trichophyton mentagrophytes; and
(ix) Epidermophyton floccosum.

For convenience, pathogens (i)-(ix) above will be referred to ‘fungal pathogens’. Accordingly, the methods described herein are for the treatment and/or prevention of infections, diseases and/or conditions caused or contributed to by any of the fungal pathogens described herein.

Diseases and/or conditions which may be treated by a method of this disclosure may include, for example, proximal subungual onychomycosis.

It should be noted that while the present disclosure and its various teachings will generally be described by reference to the treatment of fungal infections, fungal infections of the nail or onychomycosis, the disclosure further extends to methods of treating other keratinised structures and/or tissues (for example, claws, talons, hair, horns and hooves) or any form of fungal infection, including any of the infections, diseases and/or conditions described herein.

As stated, the subject to be treated using a method of this disclosure may be any human or animal subject.

The subject may be an ungulate.

The subject may be immunocompromised.

The subject may be susceptible and/or predisposed to a fungal infection.

The subject may be suffering from an onychomycosis or a fungal infection of a nail (or any other keratinous structure as desired above) and/or a disease and/or condition caused or contributed to by any of the fugal pathogens described herein.

In one teaching, a method of this disclosure may be used to treat or prevent a fungal infection, disease and/or condition occurring as a complication of a microbial infection, a genetic condition/abnormality, an allergic condition/disorder, an autoimmune disease and/or cancer.

The methods described herein all require the application of microwave energy to a subject.

Accordingly, the disclosure provides a method of treating a fungal infection, including a fungal infection of the nail, fungal infections of the unguis; onychomycosis; or Tinea unguium, said method comprising administering microwave energy to a subject.

The dose or amount of microwave energy may disrupt those fungal compounds, processes and/or pathways affecting the host immune response. In one respect, the microwave dose may be described as an indirect immunomodulatory dose. In other words, the microwave dose is formatted to disrupt (but not kill) the fungal pathogen, allowing the host immune response to recover, re-gain control and resolve the infection.

The microwave dose may be immunomodulatory in that it affects the activity, expression and/or function of various aspects of the host innate and adaptive immune response—including, for example, cytokines, chemokines, inflammatory factors, anti-inflammatory factors, apoptotic factors and the like.

As a modality electromagnetic energy, in particular energy at microwave frequencies, can be utilised to reverse a fungal pathogen's influence on the adjacent derma. Microwave energy can be controlled to be deposited in a repeatable and controlled manner to a predetermined depth or by having a consistent dosage (as described below) to enable a precise method of control. Microwave energy can also be controlled to create a pulsed hyperthermia heating effect in the adjacent derma. This effect can modulate and/or interrupt the control the pathogen exerts over local immune signalling. This technique may be utilised to restore apoptosis to renew the derma that hosts the fungus preventing advancement of the condition and removing the pathogen via natural desquamation processes. More of this discussed below.

In one teaching, this disclosure provides a method of treating or preventing an onychomycosis condition of a nail, said method comprising administering a subject suffering from, or predisposed to an onychomycosis of the nail, an immunomodulatory amount or dose of microwave energy.

Without wishing to be bound by theory, the methods described herein are based on the finding that the application of microwave energy to certain diseased tissues and/or structures can have an immunomodulatory effect. Accordingly, the methods of this disclosure find particular application in the treatment of infections, disease and/or conditions caused or contributed to by those fungal pathogens affecting or modulating the host immune response.

For example, the methods of this disclosure may be used in the treatment and/or prevention of an infection, disease and/or condition caused or contributed to by a fungal pathogen which upregulates, enhances, induces, stimulates, suppresses, downregulates and/or inhibits one or more host innate and/or acquired immune processes.

Within the context of this disclosure, the term ‘host’ refers to subjects infected with a fungal pathogen and/or suffering from a disease or condition caused or contributed to by a fungal pathogen.

Without wishing to be bound by theory, the methods of this disclosure represent an improvement over the prior art as the microwave is not applied as a thermal dose designed to directly kill, destroy, ablate or inactive the fungal pathogen, but rather as an immunomodulatory dose. The immunomodulatory does not directly kill the (fungal) pathogen.

The immunomodulatory dose does not risk damage to the cells, tissues (for example derma) and/or structures which comprise and/or surround the area (for example the keratinised structure/tissue and/or nail) to be treated. In particular, the use of an immunomodulatory dose reduces the risk of damage to the delicate nail bed.

The immunomodulatory dose may comprise microwave energy delivered in such a way that it is sufficient to modulate one or more aspects of the host immune response—in particular the immune response which might occur in the cells, tissues and/or structures (for example, the derma) associated with the nail (or other keratinised structure) to be treated.

Without wishing to be bound by theory, the fungal pathogens described herein may affect one or more aspects of the host immune response. By these mechanisms, a fungal pathogen may be able to avoid detection and/or clearance by the host immune response. By way of example, a fungal pathogen, such as a Trichophyton species (including Trichophyton rubrum) may modulate a number of host cell pathways and processes including for example, aspects of the host adaptive and innate immune system/response.

Some host cell processes, factors, pathways, compounds and/or cytokines may be down regulated by a fungal pathogen, whereas others may be upregulated.

Some of the specific cell factors, cytokines and/or compounds downregulated by fungal pathogens, for example Trichophyton Rubrum, are listed in table 1 below:

TABLE 1

| Title | Description | Function |
|---|---|---|
| IL-1β | Interleukin 1 beta | prototypic proinflammatory |
| | (leukocytic pyrogen) | cytokine, |
| | | (MAPK, CC + Apop pathway) |
| IL-6 | Interleukin 6 (interferon, | Inflammatory modulator +/− |
| | beta 2) | (TXmisReg, |
| | | JAK-STAT, PI3K pathway) |
| IL-12 | Interleukin 12 | Involved in the |
| | | differentiation of naive T |
| | | cells, as a T cell-stimulating factor |
| IL-8 | Interleukin 8 | proinflammatory CXC chemokine |
| | | (TXmisReg pathway) |
| (TNF)-α | Tumor necrosis factor | major pro-inflammatory cytokine |
| | alpha | |
| IFN-γ | Interferon gamma | critical cytokine for |
| | | innate and adaptive |
| | | immunity against infection |
| MHC | Major histocompatibility | Important for the |
| class 2 | complex (MHC) class II | presentation of antigens |
| APC | compartment | and development of an |
| | | immune response |
| CD80 | Cluster Definition 80 | immune cell activation in |
| | | response to pathogens. |
| IL-18 | Interleukin 18 (interferon- | proinflammatory cytokine |
| | gamma inducing factor) | |

Some of the specific host cell factors, cytokines and/or compounds upregulated by fungal pathogens, for example Trichophyton rubrum, are listed in Table 2 below.

TABLE 2

| Title | Description | Function |
|---|---|---|
| PI3K | Phosphoinositide 3-kinase | Intracellular signal transducer enzyme |
| p-AKT | Phosphorylated Akt | downstream effector of PI 3-kinases |
| | Protein kinase B (PKB) | (inhibition of apoptosis) |
| IL-10 | Interleukin 10 human | anti-inflammatory cytokine |
| | cytokine synthesis | that can inhibit |
| | inhibitory factor | proinflammatory responses |
| | | of both innate |
| | | and adaptive immune cells |

In view of the above, one of skill will appreciate that by downregulating those host factors involved in the host inflammatory processes and upregulating those host factors that have an anti-inflammatory effect, the pathogen is able to control the host immune response and ensure or prolong its survival.

Accordingly, the application of microwave energy (in accordance with a method of this disclosure and/or at a dose described herein) may reverse at least some of the effects a fungal pathogen has on the cells, tissues and/or structures of or adjacent to, a nail (or other keratinised structure).

The specific pathways which can be downregulated by application of microwave energy include those listed in Table 3.

TABLE 3

| Title | Description | Function |
|---|---|---|
| PI3K | Phosphoinositide 3-kinase | Intracellular signal transducer enzyme |
| p-AKT | Phosphorylated Akt | downstream effector of PI 3-kinases |
| | Protein kinase B (PKB) | (inhibition of apoptosis) |
| IL-10 | Interleukin 10 human | anti-inflammatory cytokine |
| | cytokine synthesis | that can inhibit proinflammatory |
| | inhibitory factor | responses of both innate |
| | | and adaptive immune cells |

The specific pathways which can be upregulated by application of microwave energy include those listed in Table 4.

TABLE 4

| Title | Description | Function |
|---|---|---|
| IL-1β | Interleukin 1 beta | Prototypic pro-inflammatory |
| | (leukocytic pyrogen) | cytokine, |
| | | (MAPK, CC + Apop pathway) |
| IL-6 | Interleukin 6 (interferon, | Inflammatory |
| | beta 2) | modulator +/− (TXmisReg, |
| | | JAK-STAT, PI3K pathway) |
| IL-2 | Interleukin 2 | Regulates the activities of |
| | | leukocytes/lymphocytes, |
| | | promotion of |
| | | regulatory T-cells |
| IL-12 | Interleukin 12 | Involved in the |
| | | differentiation of naive T |
| | | cells, as a T cell-stimulating factor |
| (TNF)-α | Tumor necrosis factor | Major pro-inflammatory cytokine |
| | alpha | |
| IFN-γ | Interferon gamma | Critical cytokine for |
| | | innate and adaptive |
| | | immunity against infection |
| MHC | Major histocompatibility | Important for the |
| class 2 | complex (MHC) class II | presentation of antigens |
| APC | compartment | and development of an |
| | | immune response |
| CD80 | Cluster Definition 80 | Immune cell activation in |
| | | response to pathogens. |
| CD74 | Cluster of | Involved in the formation |
| | Differentiatior 74 | and transport of |
| | (HLA class II | MHC class II peptide |
| | histocompatibility | complexes for the |
| | antigen | generation of CD4+ |
| | gamma chain) | T cell responses. |
| CD4 | cluster of | CDD4 is a co-receptor |
| | differentiation 4 | of the T cell receptor |
| T cell | T cell receptor | Surface protein complex |
| receptor | | on the T lymphocytes |
| | | responsible for recognising |
| | | antigen fragments as |
| | | peptides bound to |
| | | major histocompatibility |
| | | complex (MHC) |
| | | molecules. |
| NFkB | Nuclear Factor Kappa B | DNA binding subunit |
| | Subunit 1 | of the NF-kappa-B |
| | | (NFKB) protein complex |
| IL-18 | Interleukin 18 (interferon- | Pro-inflammatory cytokine |
| | gamma inducing factor) | |
| MAPK1 | Mitogen-activated protein | Extracellular |
| | kinase 1 | signal-regulated kinases |
| HSP | Heat shock protein | Family of proteins produced by |
| | | cells in response to exposure |
| | | to external stressors |
| MHC | Major histocompatibility | Bind and present intracellular |
| class 1 | complex class 1 | antigens |

As such, in addition to providing methods of treating or preventing fungal infections, microwave energy may be used to modulate the activity, function and/or expression of any one or more of the factors listed in Tables 1 and 2 (and 3 and 4). In this way, microwave energy can be used to disrupt, interfere with, alter and/or modulate some of the specific effects fungal pathogens exert of the host and its immune responses.

In one teaching, the disclosure provides a method of modulating Phosphoinositide 3-kinase (PI3K) expression in a subject, said method comprising applying microwave energy to the subject. In one teaching, the microwave energy may be applied to a tissue or structure which is infected with a fungal pathogen or which shows one or more symptoms associated with a fungal disease or condition. The microwave energy may be applied to a keratinised structure or tissue, for example a fingernail or toenail. The microwave energy may be applied to a nail having onychomycosis. A method of this type will result in the downregulation of PI3K expression. This method may be for the treatment or prevention of any of the fungal disease and/or conditions described herein.

The following represents a series of additional or alternate methods (methods a-k) which may be used to modulate the activity, function and/or expression of one or more host immune factors, pathways and/or responses. In each case, the method may be applied to a tissue or structure which is infected with a fungal pathogen or which shows one or more symptoms associated with a fungal disease or condition. The microwave energy may be applied to a keratinised structure or tissue, for example a fingernail or toenail. The microwave energy may be applied to an onychomycosis. The methods may be for the treatment or prevention of any of the fungal disease and/or conditions described herein.

a) The disclosure provides a method of modulating Phosphorylated Akt Protein kinase B (PKB) (p-AKT) expression in a subject, said method comprising applying microwave energy to the subject.

b) Also disclosed is a method of modulating the expression, function and/or activity of one or more cytokines in a subject, said method comprising applying microwave energy to the subject, wherein the one or more cytokines are:
IL-10
IL-1β
IL-6
IL-2
IL-12
IL-18

Method (b) may result in the downregulation of IL10 expression. Where a fungal pathogen has upregulated IL10, microwave energy may be used to restore normal levels of IL10 expression. In turn, this may repress its anti-inflammatory function in the host.

Method (b) may result in upregulation of IL-1β expression. Where a fungal pathogen has down regulated IL-1β, microwave energy may be used to restore, for example, the inflammatory and apoptosis responses mediated by this cytokine.

Method (b) may result in upregulation of IL-6 expression. Where a fungal pathogen has down regulated IL-6, microwave energy may be used to restore, for example, the inflammatory and apoptosis responses mediated by this cytokine.

Method (b) may result in upregulation of IL-2 expression. Accordingly, microwave energy may be used to stimulate or enhance the effects this important cytokine has on leukocytes/lymphocytes, the promotion of regulatory T-cells.

Method (b) may result in upregulation of IL-12 expression. Where a fungal pathogen has downregulated IL-12, microwave energy may be used to restore, for example, the effects this cytokine has on the differentiation of naive T cells and its role as a T cell-stimulating factor inflammatory and regulator/mediator of apoptosis.

Method (b) may result in upregulation of IL-18 expression. Where a fungal pathogen has downregulated IL-18, microwave energy may be used to restore, for example, the inflammatory responses mediated by this cytokine.

c) A method of modulating the expression, function and/or activity of TNF-α in a subject, said method comprising applying microwave energy to the subject.

Method (c) may result in the upregulation of TNF-α. Where a fungal pathogen has downregulated TNF-α expression, microwave energy may be used to restore, enhance or stimulate TNF-α to activate host inflammatory responses

d) A method of modulating the expression, function and/or activity of IFN-γ in a subject, said method comprising applying microwave energy to the nail.

Method (d) may result in the upregulation of IFN-γ. Where a fungal pathogen has downregulated IFN-γ expression, microwave energy may be used to restore, enhance or stimulate IFN-γ which is an important cytokine for effective innate and adaptive immune responses against infection.

e) A method of modulating the expression, function and/or activity of MHC class 1 and/or MHC class 2 in a subject, said method comprising applying microwave energy to the subject.

Method (e) may result in the upregulation of both MCH class I and II. Where a fungal pathogen has downregulated MHC class I and II expression, microwave energy may be used to restore, enhance or stimulate MHC class I and II expression; this will increase the level of antigen presentation and T/B cell activation helping to combat a fungal infection.

f) A method of modulating the expression, function and/or activity of one or more CD markers in a subject, said method comprising applying microwave energy to the nail, wherein the one or more CD markers are:
CD80
CD86
CD74
CD4

Method (f) may result in the upregulation of any one of CD80, CD86, CD74 or CD4. Where a fungal pathogen has downregulated CD80, CD86, CD74 or CD4 expression, microwave energy may be used to restore, enhance or stimulate expression of any one of these CD markers which are important in antigen recognition and/or immune cell activation.

g) A method of modulating the expression, function and/or activity of the T cell receptor in a subject, said method comprising applying microwave energy to the subject.

Method (g) may result in the upregulation of the T cell receptor. Therefore, microwave energy may be used to restore, enhance or stimulate expression of the T cell receptor in a host to facilitate antigen recognition and immune cell activation.

h) A method of modulating the expression, function and/or activity of NFkB in a subject, said method comprising applying microwave energy to the nail.

Method (h) may result in the upregulation of NFkB and microwave energy may be used to restore, enhance or stimulate NFkB expression which is important for the activation of a number of pathways which contribute to the host innate and adaptive immune response.

i) A method of modulating the expression, function and/or activity of MAPK1 in a nail, said method comprising applying microwave energy to the nail.

Method (i) may result in the upregulation of MAPK1. Accordingly, microwave energy may be used to restore, enhance or stimulate MAPK1 expression which is important for protein activation in host cells.

(j) A method of modulating the expression, function and/or activity of HSP in a subject, said method comprising applying microwave energy to the subject.

Method (j) may result in the upregulation of HSP; these are a family of proteins produced by cells in response to external stressors. These proteins can regulate certain aspects of the host innate and immune response. Microwave energy may be used to upregulate HSP expression, thereby strengthening the host immune response against a fungal pathogen.
(k) A method of modulating the expression, function and/or activity of:
(i) EGR1; or
(ii) CD79B; or
(iii) MYC; or
(iv) SOCS3;
   said method comprising applying microwave energy to the subject.

The antifungal drug terbinafine has been reported to inhibit the expression of the ERG1 and ERG11 genes. Accordingly, microwave energy can be used to achieve the same effect.

Microwave energy may be used to suppress elevated levels of CD79B(+) cells—this is important for the treatment of fungal pathogens.

Downregulation of MYC using microwave energy removes the suppression it imparts on immune surveillance and may reverse the glycolysis process which is crucial for carbon assimilation and is upregulated during infections with pathogenic fungi.

Downregulation of SOCS3 can (i) activate phagocytosis and killing of the fungal pathogen by activated dendritic cells and (ii) promote IL-6-induced tyrosine phosphorylation of STAT3 in dendritic cells to improve the capability of DCs to prime naïve T cells and initiate Th17 proliferation which protects against intracellular fungal pathogens.

In view of the above, where a fungal pathogen modulates and/or exerts a control over one or more host immune related signalling events and/or processes, a microwave-based method disclosed herein may be used to disrupt the pathogen by interrupting its influence on those host factors, allowing the host to re-establish its immune response against the fungal pathogen. For example, by modulating both host pro- and anti-inflammatory processes, a method of this disclosure may restore aspects of the host local inflammatory response. These responses help the host resolve and clear a fungal infection.

As noted above, microwave energy may be used to modulate a number of different host immune-related signalling events and processes and therefore a method of this disclosure may be used to interfere with or modulate a fungal pathogen's signalling mechanism(s). In this way, microwave energy may be used to disrupt the effect the fungal pathogen has on the host (local) immune response occurring in, for example, the derma containing the (fungal) pathogen.

In one teaching and again without wishing to be bound by theory, the microwave-based methods described herein may not directly or solely stimulate the host immune response, rather, the microwave energy may help restore the immunological balance to the disadvantage of the fungal organism.

It should be noted that while the dose is selected and applied to disrupt, interrupt or inhibit those pathogen instigated events and processes which modulate the host immune response, the dose may further induce a mild and/or localised hyperthermia in the nail (or other keratinised tissue/structure), in an associated tissue, in the derma and/or subungal tissues. The temperature elevation may be localised predominately to the surface interface of the nail derma and/or to the sub-dermal and subungal layers thereof (including all minor layers that lie within).

Microwave energy according to this disclosure may have a frequency of between about 500 MHz and about 200 GHz. In other embodiments, the frequency of the microwave energy may range from between about 900 MHz and about 100 GHz. In particular, the frequency of the microwave energy may range from about 2 GHz to about 40 GHz and in a specific embodiment has a frequency of 8 GHz.

A dose of microwave energy may comprise the application of microwave energy (to a subject to be treated) at a predetermined power rating and for some set, predetermined, time. Any given subject may be subject to repeated doses. Each repeated dose may be the same as, or different to, a previous dose. There may be rest periods between each applied dose of microwave energy.

A subject treated with repeated dose may be said to have received a ‘pulsed’ microwave treatment.

In any given dose, the microwave energy may be applied at a power of about 1 W, about 2 W, about 3 W, about 4 W, about 5 W, about 6 W, about 7 W, about 8 W, about 9 W, about 10 W, about 11 W or about 12 W. The term ‘about’ may embrace a power rating which is +/−0.5 W of any of the above listed power ratings.

In any given dose, the microwave energy may be applied to the subject (for example to a nail (or keratinised structure or tissue) for about 1 second, about 2 seconds, about 3 seconds, about 4 seconds, about 5 seconds, about 6 seconds, about 7 seconds, about 8 seconds, about 9 seconds or about 10 seconds. Within the context of time, the term ‘about’ may embrace a time which is +/−0.5 seconds of any of the above listed times.

Where a subject is to receive two or more doses of microwave energy, there may be a rest period between each dose. Each rest period may be of the same or a different duration. The rest period may last about 1 second, about 2 seconds, about 3 seconds, about 4 seconds, about 5 seconds, about 6 seconds, about 7 seconds, about 8 seconds, about 9 seconds or about 10 seconds. Within the context of a rest period, the term ‘about’ may embrace a time which is +/−0.5 seconds of any of the above listed times.

A subject may be administered one dose of microwave energy having parameters (power and duration) as specified above. A subject may be administered two or more microwave energy doses, each dose having parameters as specified above and separated from other doses by a rest period. For example, a subject may be administered about 1, about 2, about 3, about 4, about 5, about 6, about 7, about 8, about 9 or about 10 microwave doses.

Any regime described herein may be repeated over a period of days, weeks or months.

For example a particular dose (including a pulsed dose) may be re-administered every days, every 2 days, every 3, days, every 4 days, every 5 days, every 6 days, every 7 days, every 10 days or every 14 days.

In one teaching, microwave energy may be administered in accordance with the following dosage regime:
microwave energy administered at 9 watts in a “pulsed” format of 3 repetitions.

In this case microwave energy (at a power of 9 W) is administered to a subject to be treated for 3 seconds, resting for five seconds and reapplying twice (again at 9 W) for a further 3 seconds (with 5 seconds rest between subsequent doses) to achieve therapeutic and immunomodulatory doses. A subject receiving this dose may receive up to five identical treatments over a period of about 10 weeks; this roughly equates to one treatment (of 9 W, 5 s rest repeated 3 times) every 14 days.

In another teaching, a dose of microwave energy may comprise an initial dose designed to elevate the temperature of the tissue being treated and a second dose deigned to hold or maintain a specific temperature. The first dose may require the use of microwave energy at a power higher than that used for the second dose. The first dose may be applied to the subject for longer than the second dose. It should be noted that, whatever the first dose, it should not be administered for a time sufficient to cause damage either to the treatment site and/or to a keratinised structure to be treated and/or a tissue associated therewith. The first dose may not cause tissue damage.

For the first dose, the microwave energy may be used at a power of about 10 W, about 15 W, about 20 W or about 25 W.

The first dose may be administered for anywhere between about 5 and 30 seconds. For example, the first dose may be administered for about 10 seconds, about 12 seconds, about 13 seconds, about 14 seconds, about 15 seconds, about 16 seconds, about 17 seconds, about 18 seconds, about 19 seconds, about 20 seconds or about 25 seconds.

For the second dose, the microwave energy may be used at a power of about 1 W, about 2 W, about 3 W, about 4 W, about 5 W, about 6 W, about 7 W, about 8 W, about 9 W or about 10 W.

The second dose may be administered for anywhere between about 5 and 30 seconds. For example, the first dose may be administered for about 10 seconds, about 12 seconds, about 13 seconds, about 14 seconds, about 15 seconds, about 16 seconds, about 17 seconds, about 18 seconds, about 19 seconds, about 20 seconds or about 25 seconds.

A dose of this type may be modified for administration as a pulsed dose. The first pulse of doses being designed to elevate the temperature of the tissue being treated and a second pulse of doses deigned to hold or maintain a specific temperature.

A first pulse may comprise microwave energy at any of the power ratings described herein (for example 20 W) administered in short (for example 1, 2 3, 4 or 5 second) doses. Each individual dose may be separated from another by a rest period (of, for example, about 1, 2, 3 4, or 5 seconds).

The second pulse may comprise microwave energy at any of the power ratings described herein (for example (20 W) administered in short (for example 1, 2 3, 4 or 5 second) doses. Each individual dose may be separated from another by a rest period (of, for example, about 1, 2, 3 4, or 5 seconds).

The initial target temperature—i.e. the temperature to which the first dose elevates the tissue to be treated, may be in the region of about 30° C. to about 60° C., for example about 35° C., about 40° C., about 41° C., about 42° C., about 43° C., about 44° C., about 45° C., about 46° C., about 47° C., about 48° C., about 49° C., about 50° C. or about 55° C.

The secondary (or ‘hold’) temperature (i.e. the temperature at which the treated tissue is held following the first dose) may be in the region of about 20° C. to about 60° C. For example, the hold temperature may be about 41° C., about 42° C., about 43° C., about 44° C., about 45° C., about 46° C., about 47° C., about 48° C., about 49° C., about 50° C. or about 55° C.

Any of the dose regimes described herein may be applied directly to the site of infection. Where the regime includes repeat doses, each dose may be applied to the same site. Each dose may be administered to a different site. The dose administration site may be selected from (i) the site of the fungal infection (for example a nail or other keratinised tissue or structure); (ii) a site proximal or adjacent to a fungal infection; (iii) a site distal to the site of a fungal infection. By way of example, one or more (for example some or all) of the doses making up a subject's treatment regime may be applied to healthy tissue, for example healthy tissue near, proximal or adjacent to the area, tissue or (keratinised) structure to be treated.

In one teaching, the methods described herein may exploit microwave energy administered (at any stated dose or in accordance with any of the disclosed regimes) to a fixed spot, one or more times before moving to an adjacent treatment area. In this way, over time and via a series of doses, the entire surface of a structure or tissue (for example a nail) to be treated may be administered a sufficient dose of microwave energy.

Alternatively, a method of this disclosure may involve administering the microwave energy to a specific location on a structure or tissue (for example a nail) to be treated and moving a microwave applicator in a controlled seeping motion to deliver and spread the energy. Over

Also disclosed is a method of improving the treatment or prevention of a disease or condition caused or contributed to by a fungal pathogen, said method comprising hydrating the area to be treated before applying microwave energy.

The microwave energy may be applied in accordance with any of the methods described herein and/or at any of the described dose(s).

Any of the methods disclosed herein may further include mycology testing steps in which tissues and/or keratinised structures are sampled or swabbed before, during and/or after microwave treatment in order to determine to what extent the fungal pathogen has been inactivated or affected by the microwave energy treatment.

The methods of this disclosure may be further supplemented with dose regimes designed to maintain a level of treatment and/or prevention. In other words, once a fungal infection has been resolved (by application of a method and/or dose of microwave energy described herein), microwave energy, again at a dose described herein, may be used to prevent recurrence of the infection.

The area to be treated may comprise a nail (for example a fingernail and/or a toenail) which is infected with a fungus. The improved method of this disclosure may therefore comprise the step of rehydrating, hydrating or moistening a nail before treatment with microwave energy.

Without wishing to be bound by theory, fungal infections can cause nails to become brittle and dry. Additionally or alternatively, a fungal infection may cause the distal nail to detach from the nail bed, creating an air-gap under the nail. When microwave energy is applied to an infected (and dry, brittle, detached) nail, that energy may readily permeate into the surrounding tissues/derma and/or underlying tissues of the nail bed. By moistening and/or hydrating the nail, any applied microwave energy becomes constrained to the nail region with less of the energy permeating into the surrounding tissues/derma and/or underlying tissues of the nail bed.

A nail may be moistened or hydrated in order to produce a ‘wet’ nail for treatment.

Any suitable hydration and/or lossy ionic fluids or materials may be used to produce a wet nail. Fluids of this type shall be referred to herein after as ‘hydration fluids’. A hydration fluid may be brought into contact with a nail to be treated and left (or incubated on or with the nail and/or its surrounding tissues) for a period of time suitable to generate a ‘wet’ nail. Without being bound by theory, an hydration fluid may function to buffer the energy closer to the surface or within certain predetermined or defined regions of the nail structure and/or its surrounding tissues and structures. Additionally or alternatively, an hydration fluid for use may be have low microwave loss properties. Examples of such low microwave loss fluids may include, for example, chilled fluids, distilled and/or de-ionized water; fluids of this type may be used to limit the heating effect of the microwave energy in regions of the nail, its surrounding tissues and/or structures which are intended to be protected or preserved.

A fluid with a high salinity can be administered to, around and/or under the nail to help constrain or further constrain the energy within (or substantially within) the nail structure. Where there is an air-gap between the nail and the nail bed (caused by, for example, a fungal infection in the nail) a microwave energy based treatment may be further improved by the application of Sodium chloride (NaCl) to the nail. For example, a saline solution may be injected or deposited under the nail to take up the air gap. Additionally or alternatively, a saline solution may be injected or deposited under and around the nail to take up the air gap—this approach may be particularly useful where the nail is ‘wet’ (e.g. moist and/or has been hydrated).

In one teaching, a water bath may be used to moisten nails prior to treatment.

A nail may be pre-treated so that, in comparison to a nail which has not been pre-treated, that nail better retains moisture. A suitable pre-treatment may comprise the application of a quantity of urea (for example Urea at a concentration of about 10% to about 50% (for example about 15%, about 20%, about 25%, about 30%, about 35%, about 40% or about 45%) prior to any moisture or hydration treatment.

Any pre-treatment may be applied to a nail at least one or more days prior to any moisture or hydration treatment is to be applied to a nail.

Without wishing to be bound by theory, the dielectric constant and electrical loss tangent increase with increased moisture content. In turn, this increases the heating and focuses the energy within a smaller zone—the effect is to substantially or better confine the microwave energy treatment to the nail and the surrounding area. This enhances the energy density and reduces heating of the underlying tissue which makes for a more easily tolerated treatment without the requirement for local anaesthetic. It also reduces the risk of damage to off (or non-) target tissues and structures.

In one embodiment, the means for administering or delivering the microwave energy to a subject to be treated (for example in accordance with one or more of the methods described herein) comprises an applicator formed, adapted and/or configured to deliver or administer microwave energy to the subject.

The means for delivering or administering microwave energy may electrically match the range of epsilon relative (relative permittivity) values of the tissue or structure (for example a nail, toenail or other keratinised structure) affected by, or infected with a fungal pathogen. In this way, it is possible to ensure efficient delivery of the microwave energy to the tissue/structure.

Advantageously, the means for administering the microwave energy to a subject may comprise a component or part for contact with a subject to be treated. The part or component for contact with the subjected to be treated may be removable such that it can be discarded or sterilised after use.

In one embodiment, the means for administering the microwave energy may comprise a single application element or a hand piece which accepts a removable tip; this can be a single use, disposable component or a reusable component intended to be sterilized between uses. Advantageously, the part or component for contact with the subject to be treated may comprise a reuse mitigation function to prevent accidental or attempted reuse.

In one embodiment, the part or component for contact with the subjected to be treated may be shaped, formed or adapted so as to be compatible with a particular body part, structure, surface or contour thereof. For example, the part or component may comprise a thin, curved or round surface, compatible with the physical properties or profile of an internal or external body part or a surface thereof, including, for example the nail, or the sinus unguis.

The contacting component part may also be lubricated or be coated or used with a buffer medium to remove airgap discontinuity between the contacting part and the nail to ensure good delivery of energy, for example propylene glycol, glycerine.

The means for delivering or administering the microwave energy to a subject may be connected to the microwave source via a flexible cable. In one embodiment the means for delivering or administering the microwave energy to a subject (i.e. the applicator) may be connected to the microwave source via a flexible cable with locking connections having both microwave and signal data cables and may be reversible to enable connection to either port.

In one teaching the disclosure provides an apparatus for delivering or administering microwave energy to keratinised tissues or structures, particularly tissues or structures as described herein which exhibit symptoms of any of the diseases described herein; the apparatus comprising:
a microwave source for providing microwave energy, connectable to a system controller for controlling at least one property of the microwave radiation provided by the microwave source; and
a monitoring system for monitoring the delivery of energy and an applicator means, for example an applicator device, for delivering or administering microwave energy;
wherein: the applicator is configured to deliver precise amounts of microwave energy provided by the source at a single frequency or across a range of frequencies.

This system may be further integrated to include all components including energy generation and delivery components within a single hand-piece.

In one teaching, any of the methods described herein may be cosmetic methods used to improve the appearance of a subject's keratinised structures, for example their nails, fingernails and or toenails. A cosmetic method of this disclosure is especially useful where the appearance of a subject's nails has been affected by a fungal pathogen and/or a diseases and/or conditions caused or contributed to by a fungal pathogen. Any of the methods described herein (which may be re-purposed as cosmetic methods) may be repeated a number of times until a cosmetically beneficial or acceptable improvement in the appearance of the keratinised structure (for example the nail or the like) is obtained.

Any of the methods described herein may be supplemented by methods or procedures which induce reactive hyperemia, wherein blood perfusion after a period of enforced low perfusion increases to a level higher than before the intervention. By way of example, pressure could be applied to the region to be treated (for example the toe or finger) to restrict blood perfusion before application of the microwave energy. As or after the microwave energy is or has been administered, the pressure may be released and resulting increased perfusion would provide a cooling effect. This would restrict and limit tissue damage caused by any heating effect associated with the administration of microwave energy.

Any of the methods described herein may be supplement and/or improved by or with the use of a temperature sensitive material or thermochromic barrier or coating. A material, barrier or coating of this type may change colour upon exposure to heat as might be induced by a microwave treatment. Moreover a temperature sensitive material or thermochromic barrier and/or coating may be used a way to monitor which areas of a site to be treated have been administered microwave energy—those sites administered microwave energy being indicated by a change of colour in the temperature sensitive material and/or thermochromic barrier and/or coating.

A temperature sensitive material or thermochromic barrier and/or coating may be applied to a (keratinised) tissue or structure to be treated. The material, barrier and/or coating may be applied to all or part of a site (for example a nail, toenail or fingernail) to be treated.

The temperature sensitive material, thermochromic barrier or coating may take the form of a thermochromic paint, a thermochromic film or thermochromic paper.

The temperature sensitive material or thermochromic barrier and/or coating may be calibrated to change colour upon contact with a particular dose of microwave energy and/or upon expose to a particular temperature. For example, a temperature sensitive material or thermochromic barrier and/or coating may change colour in response to a temperature rise brought about by a rise in temperature in a tissue treated with or administered, microwave energy.

A thermochromic barrier and/or coating or temperature sensitive material may find particular application when a treatment area is relatively large and a microwave applicator has to be applied to several distinct positions in order to cover the full area. In such cases, a thermochromic barrier and/or coating or temperature sensitive material will allow the user to monitor and keep track of, those areas of the treatment site which have been exposed to the microwave energy dose and those areas which have not.

### BRIEF DESCRIPTION OF THE DRAWINGS

FIG. 1 (upper and lower panels). Nail comprises the nail plate (corpus unguis) 1, the Lateral horns (Lunula) 2, the nail root (germinal matrix), (radix unguis) 3, the lateral nail fold (Paronychium), 4, the quick (Hyponychium) 5, the nail bed (sterile matrix) 6, the cuticle (Eponychium) 7, the nail cleft (sinus unguis) 8 the periosteum 9 and the ventral floor 10.

FIG. 2 depicts a microwave power generator system for medical applications. The apparatus comprising: —a microwave source for providing microwave energy 11, connectable to a system controller 12 for controlling at least one property of the microwave radiation provided by the microwave source; and a monitoring system 13 for monitoring the delivery of energy and an interconnecting cable 14 and an applicator hand piece 15 and a removable applicator means 16, for example an applicator device, for delivering microwave energy, wherein: —the applicator is configured to deliver precise amounts of microwave energy provided by the source at a single frequency or across a range of frequencies.

FIG. 3 depicts a schematic representation of a cellular signalling system. In this illustration the main immune interactions between the host and Trichophyton Rubrum 17 are summarised.

FIG. 4 shows the components of an apparatus according to the present disclosure, the components shown separately for ease of reference. The apparatus comprises a generator system 24 with a locking microwave connection 25 to a flexible microwave cable 26 connected to a hand piece 27 (which may have the same type of locking connection) which accepts an applicator component 28.

FIG. 5 shows an embodiment of a microwave treatment for Onychomycosis. In this embodiment a microwave applicator 29 is introduced above the nail plate (corpus unguis) and microwave energy is applied to the nail structure producing targeted tissue irradiation 30 to a depth 31 determined by the frequency of application in conjunction with the properties of the nail/tissue structure. The microwave frequency chosen will be sufficient that the penetration of energy will be limited to the to prevent damage to the underlying tissues such as the nail bed (sterile matrix) 6, and the periosteum 9.

FIG. 6 depicts an Onychomycosis infection which covers a significant proportion of the nail plate 32, various treatment application regimens may be used to cover the required area of the infected nail. This may be done as a piecewise application 33 where overlapping treatment areas may be selected. This may be further enhanced by using a thermochromic barrier or coating 34 that may be affixed to the nail to indicate regions already treated. Similarly, the treatment may comprise a continuous sweeping motion from left to right and reversed from right to left 35 or from proximal to distal and reversed from distal to proximal 36.

FIG. 7 depicts an Onychomycosis infection which covers a significant proportion of the nail plate, various treatment application regimens may be used to cover the required area of the infected nail. For example, the piecewise application 37 may occur from proximal to distal in a series of linear steps 38 or in a series of linear sweeps 39 each in the same left to right or right to left direction 40 or as a series of linear sweeps 41 from proximal to distal 42 or from distal to proximal.

FIG. 8 depicts a number of simulated results (COMSOL finite element analysis) are presented for various nail properties.

FIG. 9 depicts a number of simulated results (COMSOL finite element analysis) are presented for various nail properties.

FIG. 10 depicts Trichophyton rubrum spores were cultured in agar petri dishes.

FIG. 11 depicts Trichophyton rubrum spores were cultured in agar petri dishes after microwave (MW) treatment.

FIG. 12 is a graph illustrating the mean area (mm²) of clearance in culture following microwave treatment for each protocols.

FIG. 13 depicts an Onychomycosis case prior to treatment with microwave energy.

FIG. 14 depicts the Onychomycosis case of FIG. 13 1 month after treatment with microwave energy.

FIG. 15 depicts the Onychomycosis case of FIG. 13 6 month after treatment with microwave energy.

FIG. 16 depicts the Onychomycosis case of FIG. 13 10 month after treatment with microwave energy.

FIG. 17 depicts the Onychomycosis case of FIG. 13 10 month after treatment with microwave energy, specifically distal outgrowth of the nail.

### DETAILED DESCRIPTION OF THE INVENTION

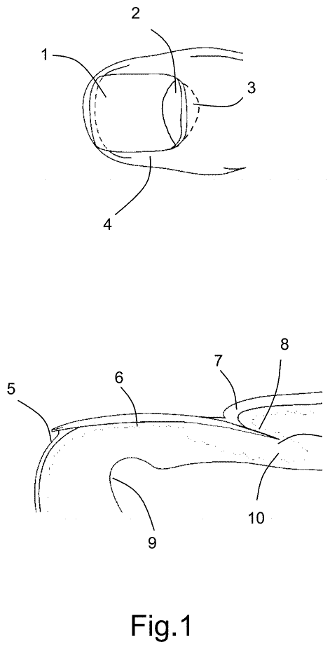

The anatomy of the nail is illustrated in FIG. 1 (upper and lower panels). Nail comprises the nail plate (corpus unguis) 1, the Lateral horns (Lunula) 2, the nail root (germinal matrix), (radix unguis) 3, the lateral nail fold (Paronychium), 4, the quick (Hyponychium) 5, the nail bed (sterile matrix) 6, the cuticle (Eponychium) 7, the nail cleft (sinus unguis) 8 the periosteum 9 and the ventral floor 10.

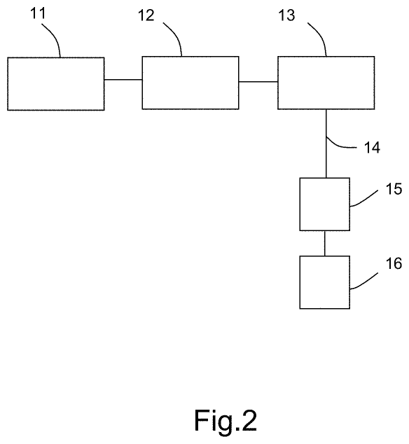

An embodiment of a microwave power generator system for medical applications is illustrated in FIG. 2 The apparatus comprising: —a microwave source for providing microwave energy 11, connectable to a system controller 12 for controlling at least one property of the microwave radiation provided by the microwave source; and a monitoring system 13 for monitoring the delivery of energy and an interconnecting cable 14 and an applicator hand piece 15 and a removable applicator means 16, for example an applicator device, for delivering microwave energy, wherein: —the applicator is configured to deliver precise amounts of microwave energy provided by the source at a single frequency or across a range of frequencies.

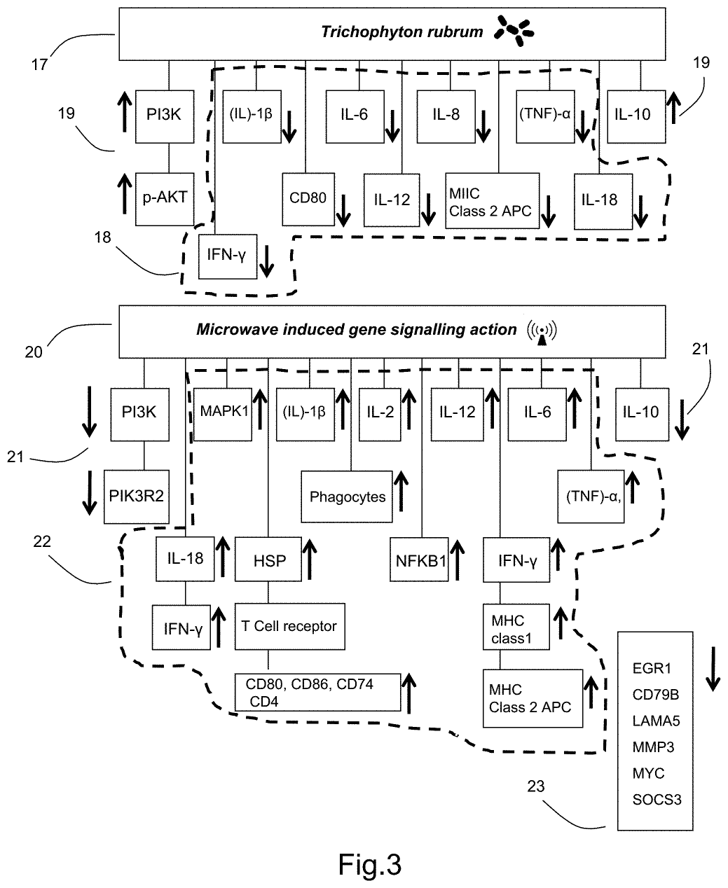

FIG. 3 provides a schematic representation of a cellular signalling system. In this illustration the main immune interactions between the host and Trichophyton rubrum 17 are summarised.

The intracellular enzyme Phosphoinositide 3-kinase PI3-K has been linked to a range of intracellular functions for example, cell growth, differentiation, proliferation, motility, survival and trafficking. PI3-K typically activates protein kinase B (PKB, aka Akt) in the PI3-K/AKT/mTOR pathway. PI3K/Akt is also known to play an important role in mediating survival of monocytes/macrophages in response to signalling factors and cytokines and in blocking apoptosis by toxic invading organisms. PI3K increases anti-inflammatory cytokine production and has also been known to offer a mediated protection against epithelial damage during infection by some fungal species for example Candida albicans [′] and has been implicated in the anti-apoptotic property of a variety of pathogens. For example, sustained PI3K/Akt signalling [ⁱⁱ] in cells infected with human pathogen Aspergillus fumigatus can exert a cytoprotective effect enabling infected macrophages to resist apoptosis. Although the evidence for this occurring with Trichophyton rubrum 19 is limited, modulation of this pathway is anticipated based upon its involvement with other fungal pathogens.

Referring to the lower panel in FIG. 3, microwave energy 20 applied to tissue infected with a fungal pathogen, may cause a down regulation of (PI3-K) 21 which regulates cellular signalling. This may be achieved by any of the microwave energy doses described herein, including the pulsed or continuous microwave energy-based methods. An inhibitor of PI3K will decrease anti-inflammatory cytokine production, and this downregulation has been achieved with the use of microwave energy downregulating the PI3K/PIK3R2 signalling pathway.

It has also been observed [Error! Bookmark not defined.] in patients with chronic widespread dermatophytosis (CWD) due to Trichophyton rubrum that their macrophages secreted lower amounts of pro-inflammatory cytokines, including interleukin (IL)-1β, IL-6 and tumor necrosis factor (TNF)-α 18, however these patients also expressed increased levels of anti-inflammatory cytokine IL-10.

The signalling and/or expression of these cytokines can be modulated (for example, reversed) by the application of microwave energy, this upregulates at least interleukin (IL)-1β, IL-6, ad tumor necrosis factor (TNF)-α, and down-regulates expression of the anti-inflammatory cytokine IL-10.

Trichophyton rubrum has also been seen [iii] to act as a downregulator of class II major histocompatibility complex (MHC) antigens 18 and in the expression of co-stimulatory molecules. It has now been shown that MHC antigen expression and Interleukin (IL)-18 expression (an immune-enhancing cytokine, which induces Interferon (IFN)-γ which in turn activates macrophages) can be upregulated by administration of microwave energy as disclosed herein. It should be noted that upregulation of IFN-gamma is linked to the up-regulation of MHC class I expression and has a positive effect on antigen processing and presentation. Microwave energy (via modulation of IFN-gamma) has also been shown to have an effect on MHC class II expression induces.

IFN-γ has been shown [iv] to impair Trichophyton rubrum proliferation through the production of reactive oxygen species (ROS). Moreover, IL-12 and IFN-γ are influential in controlling the infection and both these factors and have now been shown to be upregulated by microwave energy for therapeutic benefit.

In observed [v] interactions between Trichophyton rubrum conidia with macrophages, the expression of (TNF)-α and IL-10 were modulated (but not IL-12 and nitric oxide). Infected macrophages downregulated the expression of co-stimulatory molecules (CD80 and CD54). Microwave energy application can increase expression of heat shock proteins (HSPs) enhancing T-cell activation resulting in subsequent upregulation of CD80, CD86, CD74 and CD4. Dermatophytes including Trichophyton rubrum can also intrinsically express HSP70 and HSP90 in response to physiological stresses [vi, vii] and HSP90 plays a crucial role in the development of antifungal resistance of Candida albicans to both of the main antifungals, the echinocandins and the azoles. Since it has been shown that microwave energy induces an HSP response in tissue it may also be used to induce a similar response in the dermatophyte with this being used to exhaust the protective influence of the dermatophyte's expression of HSP rendering the pathogen susceptible to the host's natural immune system defences. Microwave energy applied in brief pulses or for short intervals represents a unique and unnatural challenge to any organism and may be one that its evolved protection mechanisms are unequipped to combat. Accordingly, a method provided by this disclosure may exploit microwave energy as a way to upregulate expression of HSP70 and HSP90 in certain fungal pathogens. A method of this type may comprise administering microwave energy to any of the subjects described herein at a dose described herein.

Trichophyton rubrum has also been determined [viii] to increase the expression of TLR2, TLR4 and TLR6 and to induce HBD-1 and HBD-2 production. The innate immune function of keratinocytes as the initial stage of localised skin immunity is effectively influenced and adapted by Trichophyton rubrum ensuring the pathogen can become established and the infection can persist. Microwave energy may be used to modulate the function of keratinocytes and to reverse or counter the effects the dermatophyte has on the innate immune system.

The common antifungal drug terbinafine has been reported to inhibit the expression of the ERG1 and ERG11 genes [ix]. The ERG1 gene can be downregulated by microwave energy 23.

Increased CD79b(+) cells have been observed in feline models [x] with significant expression of class II antigens of the major histocompatibility complex associated with epithelium involvement near to fungal elements. Significantly, more CD79b(+) cells were found to have infiltrated the epithelium. Elevated levels of CD79B have also been reversed by application of microwave irradiation 23.

MYC [xi] is known to suppress immune surveillance, MYC [xii] also plays a primary role in the regulation of aerobic glycolysis. MYC directly activates the transcription of the majority of glycolytic genes. The glycolysis pathway is crucial for carbon assimilation and is upregulated during infections with pathogenic fungi such as C. albicans [xiii], Trichophyton rubrum [xiv] and Paracoccidioides brasiliensis [xv] It is known [xvi] that macrophages react to increased glycolysis by upregulating antimicrobial inflammation and by killing pathogens. In the case of Candida-macrophage interactions a combined metabolic change occurs, with concurrent upregulation of glycolysis in both host and pathogen establishing competition for glucose. As Candida can survive on multiple carbon sources it has an advantage over infected macrophages which are metabolically constrained to glycolysis and depend upon a glucose source for survival. Candida benefits from this constraint by exhausting the supply of available glucose thereby starving the macrophage of energy and establishing and advantage. Microwave energy 23 can be used to downregulate MYC 23 which may be used to reverse the glycolysis process local to dermatophyte infection.

It has been shown [xvii] that the suppression of SOCS3 in dendritic cells can enhance the activation and specialisation of dendritic cells. Suppression of SOCS3 can activate phagocytosis and killing of the fungal pathogen by the dendritic cells. Suppression of SOCS3 can also increase promote IL-6-induced tyrosine phosphorylation of STAT3 in dendritic cells and improved capability of DCs to prime naïve T cells and initiate Th17 proliferation which protects against intracellular fungal pathogens. Microwave energy has now been demonstrated to supress SOCS3 by a factor of almost 6× in tissue with elevated expression of this gene 23.

Another mechanism that may be exploited is the Ca2+ calcium signalling pathway [xviii] which can theoretically be modulated using pulsed application of microwave energy.

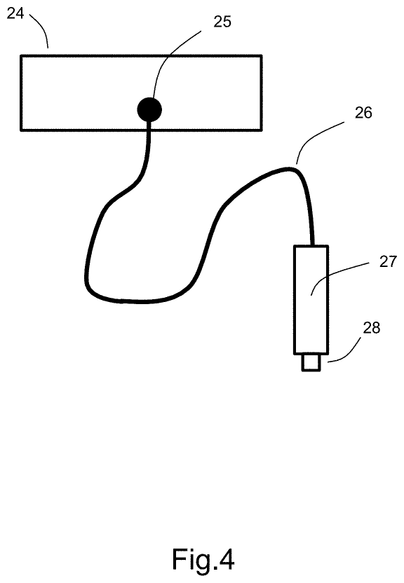

FIG. 4 shows the components of an apparatus according to the present disclosure, the components shown separately for ease of reference. The apparatus comprises a generator system 24 with a locking microwave connection 25 to a flexible microwave cable 26 connected to a hand piece 27 (which may have the same type of locking connection) which accepts an applicator component 28. The applicator component is designed to match to the combined tissue and material dielectric properties of the nail, pathogen infection and the nail bed 6. The cable 26 may include both microwave and signal data cables and may be reversible to enable connection to either port.

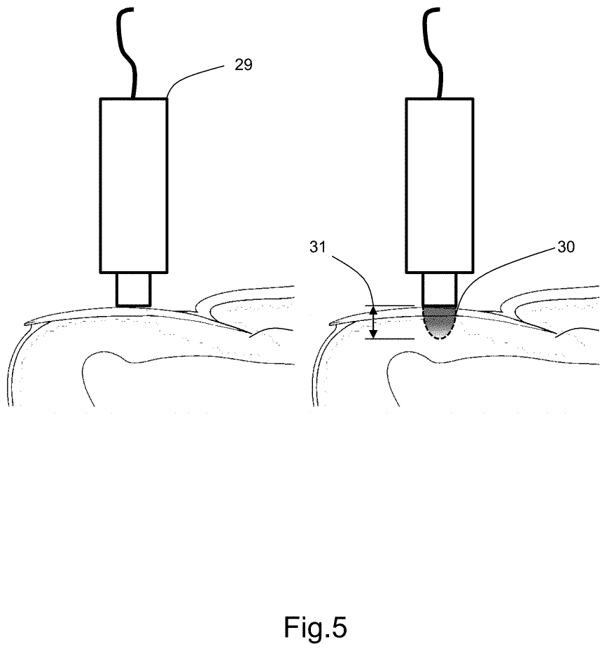

FIG. 5 shows an embodiment of a microwave treatment for Onychomycosis. In this embodiment a microwave applicator 29 is introduced above the nail plate (corpus unguis) and microwave energy is applied to the nail structure producing targeted tissue irradiation 30 to a depth 31 determined by the frequency of application in conjunction with the properties of the nail/tissue structure. The microwave frequency chosen will be sufficient that the penetration of energy will be limited to the to prevent damage to the underlying tissues such as the nail bed (sterile matrix) 6, and the periosteum 9.

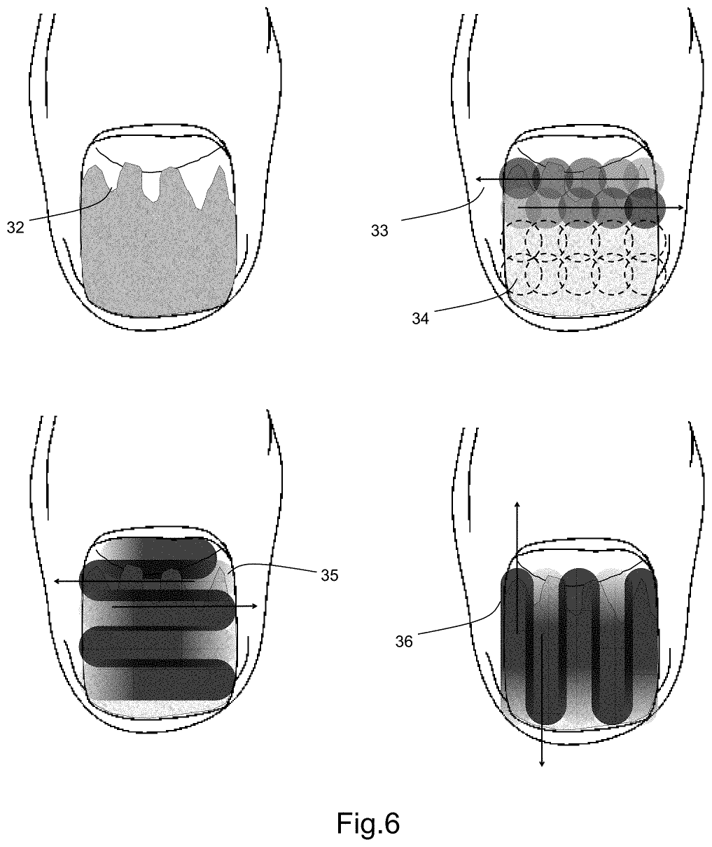

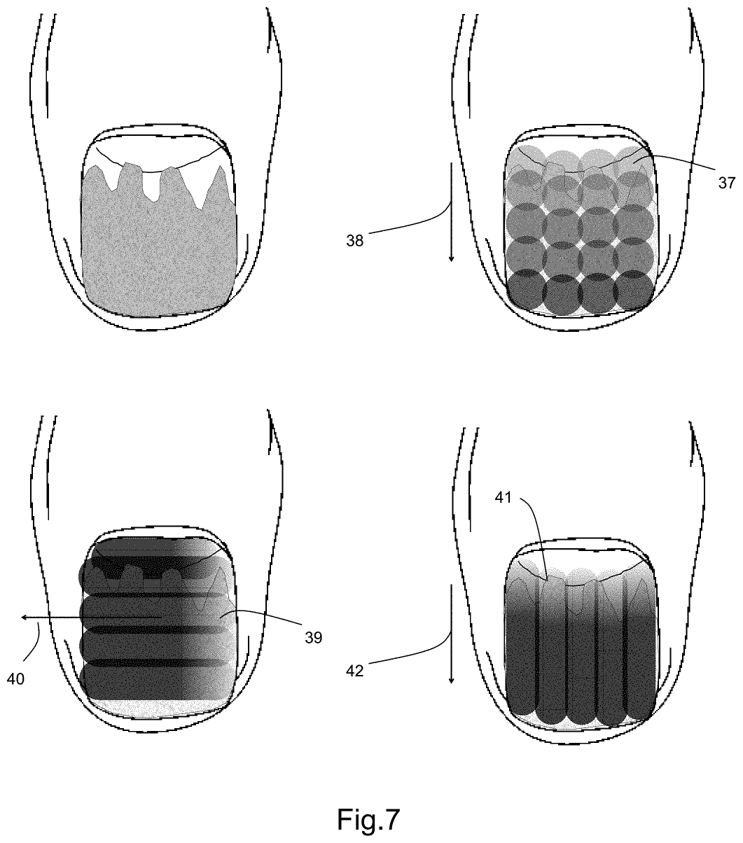

In reference to FIGS. 6 and 7, where the Onychomycosis infection covers a significant proportion of the nail plate 32, various treatment application regimens may be used to cover the required area of the infected nail. This may be done as a piecewise application 33 where overlapping treatment areas may be selected. This may be further enhanced by using a thermochromic barrier or coating 34 that may be affixed to the nail to indicate regions already treated. Similarly, the treatment may comprise a continuous sweeping motion from left to right and reversed from right to left 35 or from proximal to distal and reversed from distal to proximal 36. Alternatively, the piecewise application 37 may occur from proximal to distal in a series of linear steps 38 or in a series of linear sweeps 39 each in the same left to right or right to left direction 40 or as a series of linear sweeps 41 from proximal to distal 42 or from distal to proximal. This application method may be further enhanced by utilising materials or compounds applied to the nail with lubricants embedded or added to reduce friction and facilitate this sweeping motion. It should be noted that any sweeping motion of an applicator across the surface of a nail to be treated, may be facilitated by the use of a lubricant. For example a lubricated film, barrier, fluid or compound may be coated, attached and/or deposited onto the surface to be treated (for example the surface of a nail, toenail or fingernail) to enhance travel of an applicator across the that surface.

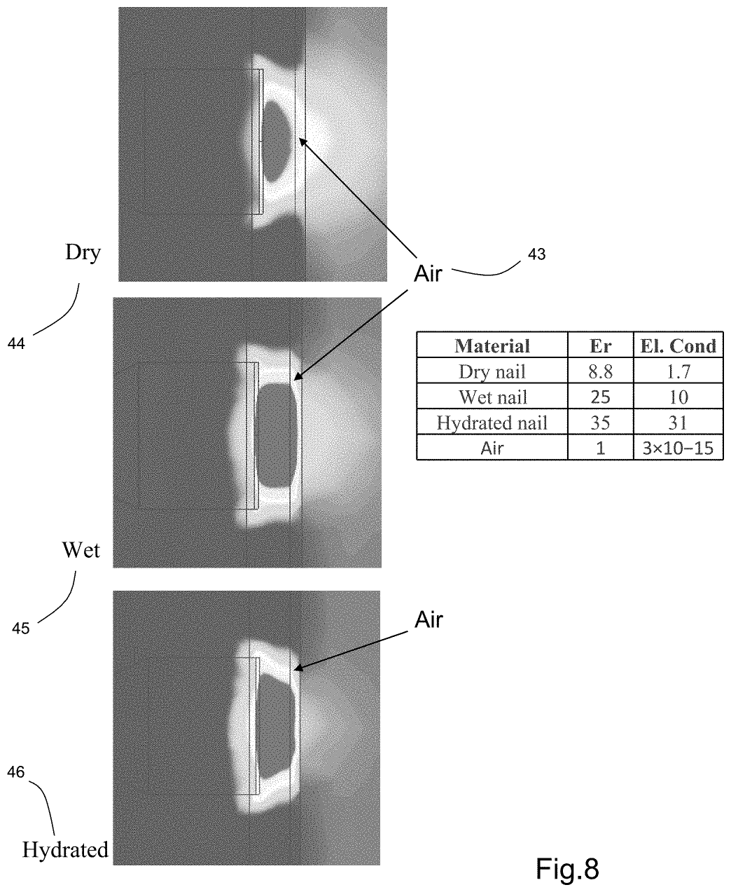

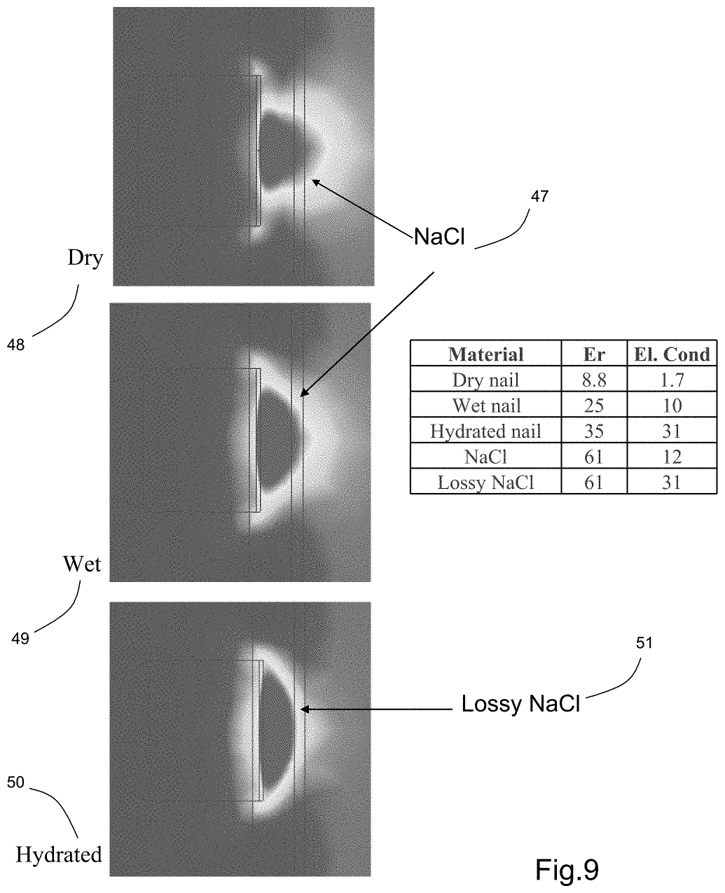

In FIG. 8, a number of simulated results (COMSOL finite element analysis) are presented for various nail properties. Firstly, a nail with an underlying air-gap 43 is considered. This often occurs in an Onychomycosis infection where the distal nail detaches from the nail bed due to the underlying fungal invasion. In this scenario it can be seen in the case of a dry nail 44 that applied microwave energy can permeate more readily into the underlying tissues of the nail bed. Where the nail has been moistened 45 it can be seen via simulation that the energy can be further constrained within the nail region. This is further developed for a hydrated 46 nail where the energy is further constrained to this region with less energy permeating into the underlying tissue. The wet nail may be achieved by use of a water bath to moisten the nails prior to treatment. In the case of hydrated nail this may be achieved by application of urea (10-50%) prior to the treatment (one or more days before) to increase the retained moisture within the nail. In each case the dielectric constant and electrical loss tangent increase with increased moisture content. This increases the heating and focuses the energy within a smaller zone within and proximate to the nail. This enhances the energy density and reduces heating of the underlying tissue which makes for a more easily tolerated treatment without the requirement for local anaesthetic.

This approach can also be enhanced by the application of Sodium chloride (NaCl) 47 to each nail example. In the case of the dry nail 48, saline may be injected or deposited under the nail to take up the air gap. In the case of the wet nail 49 saline may be injected or deposited under and around the nail to take up the air gap. In the case of the hydrated nail, fluid with a high salinity can be added under the nail to constrain the energy significantly within the nail structure. These methods may have advantages where the Onychomycosis infection results in dry or brittle nails. In those cases, the energy may pass through the nail and fungal regions into the nail bed causing excessive heating of the derma tissues rather than the desired fungal infected regions. In this regard, hydration and/or lossy ionic fluids or materials may be employed to buffer the energy closer to the surface or within desired regions of the nail structure. In addition, low microwave loss fluids such as distilled or de-ionized water (including chilled fluids) can conversely be used to limit the heating effect of the microwave radiation in regions intended to be protected or preserved.

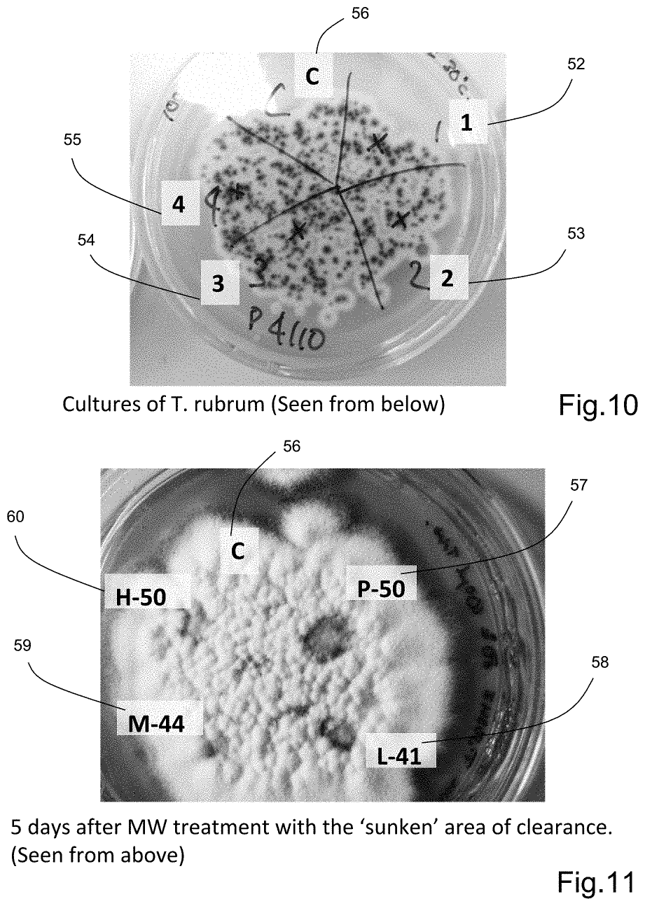

In order to examine the direct effect of microwave energy on Trichophyton rubrum fungus, Trichophyton rubrum spores were cultured in agar petri dishes as shown in FIG. 10. Microwave energy at 8 GHz was applied to the culture from below using the SWIFT microwave system. A number of energy application protocols were trialled and the culture was monitored for 10 days to document reaction to this energy. There were 4 distinct areas/segments of energy application each receiving energy with different protocol parameters. From the underside the 1^{st} region 52 corresponded to H-50 on the top-side 60, the 2^{nd} underside region 53 corresponded to M-44 on the top-side 59, the 3^{rd} underside region 53 corresponded to L-41 on the top-side 58 and the 4^{th} underside region 55 corresponded to P-50 on the top-side 57. There was also a control region C 56 which did not receive any energy.

In the photograph in FIG. 11 taken 5 days pending application of the energy a distinctive sunken area within the culture can be seen where the Trichophyton rubrum fungus has been disrupted from normal growth compared to the control region C 56.

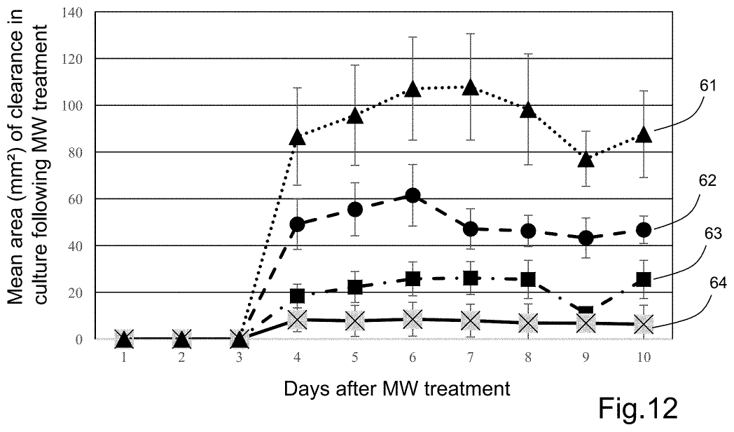

The area of clearance was recorded each day and was found to peak approximately 6 to 7 days after the treatment before regressing due to fungal ingrowth from the treatment margin. It should be noted that no new growth occurred from within the treated zone so it was determined that untreated fungus at the margin regained this area as opposed to a recovery occurring inside the treated zone. This data is presented in FIG. 12 which illustrates the mean area (mm²) of clearance in culture following microwave treatment for each of the protocols. This data is further summarised in a table below.

| Ref | | | T1 | | T2 | |
|---|---|---|---|---|---|---|
| number | Regimen | Ramp | (° C.) | Hold | (° C.) | Segment |
| 62 | H-50 | 15 W 20 s | 55 | 5 W 30 s | 49-50 | 1 |
| | (circle) | | | | | |
| 63 | M-44 | 15 W 13 s | 46-47 | 5 W 20 s | 44 | 2 |
| | (square) | | | | | |
| 64 | L-41 | 20 W 10 s | 41 | 5 W 20 s | 41-42 | 3 |
| | (cross) | | | | | |
| 61 | P-50 | (20 W | 42 | (20 W | 48-50 | 4 |
| | (triangle) | 3 s) × 3 | | 5 s) × 3 | | (pulsed) |
| | Control | n/a | n/a | n/a | n/a | C |

Note:
H-50 was 15 Watt/20 sec ramp 5 Watt/30 sec hold 62 (circles) applied to segment 1.
M-44 was 15 Watt/13 sec ramp 5 Watt/20 sec hold 63 (squares) applied to segment 2.
L-41 which was 20 Watt/10 sec ramp 5 Watt/20 sec hold 64 (crosses) applied to segment 3.
P-50 which was 20 Watt/3 sec pulsed 3× times with a 20 Watt/5 sec hold pulsed 3× times 61 (triangles) applied to segment 4.

Temperatures were balanced to establish if there was a difference between pulsed or continuous energy application. It was found that for the same temperature during the hold phase that intermittently pulsed (seconds) microwave energy had a significant effect on the Trichophyton rubrum colony morphology resulting in the highest amount of clearance area. The energy levels required to achieve this also included the heating of the agar medium which necessitated higher power levels that could not be expected for treatment in human tissue due to the volume of material and distance travelled by the energy in the experimental media. The temperature levels however were translatable for human use.

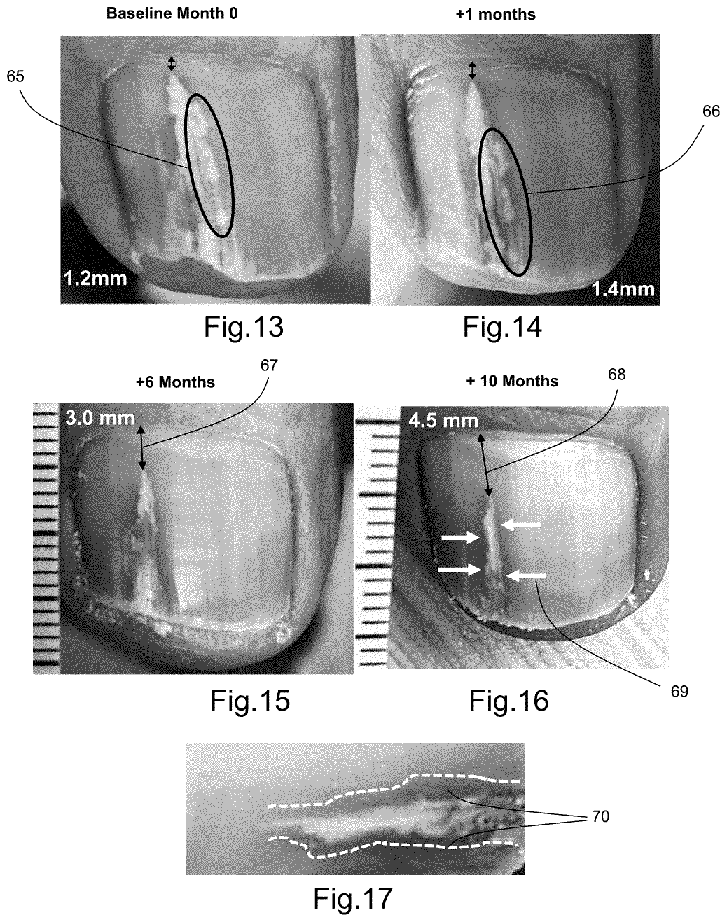

As illustrated in FIG. 13 this treatment was applied to an Onychomycosis case by an independent clinician who secured the patients' approval to treat only with microwave energy. The baseline condition state was recorded in FIG. 13, In this case a “nail spike” of Onychomycosis infection was presented. The initial distance recorded between the infection and the proximal nail fold was 1.2 mm. In this state left to progress it could reasonably be assumed that without treatment the infection would eventually engulf the entire nail area including the proximal nail fold resulting in the destruction of the nail.

A distinctive tri-globular area 65 was observed as a focal point to monitor the progression of the infection. Pending treatment and upon follow-up at +1 month as illustrated in FIG. 14, the tri-globular area 66 was observed to have advanced towards the distal end of the nail with the area of separation between the advancing front of the Onychomycosis invasion and the proximal nail fold increasing to 1.4 mm.

Further treatment was provided and pending a long treatment hiatus the patient presented+6 months from the initial baseline with an improved state of the Onychomycosis condition of the nail. This is illustrated in FIG. 15 where a 3 mm separation 67 between the proximal nail fold and the advancing front of the Onychomycosis infections was evident. Interestingly a darker lateral margin was observed to have appeared between the healthy nail bed and the region of Onychomycosis and this was not observable in the baseline photographs.

The Patient received further treatment and returned+10 months from baseline with further improvement evident. In this observation the separation 68 between the proximal nail fold and the advancing front of the Onychomycosis infection increased to 4.5 mm of clear new nail. This trend would suggest that the nail should continue to grow out naturally and thus eliminate the fungal infection. It was however interestingly noted that the lateral margin 70 had advanced in the medial direction 69 in addition to the distal outgrowth of the nail as highlighted in FIG. 17. It must be assumed that the host immune system had mounted a response to the fungus and thus had succeeded in eliminated an area of fungal invasion in addition to preventing the advancement of the disease towards the proximal nail fold. The dark (red) margins 70 are suggestive of an inflammatory or reactive region that according to the aforenoted theory could be suppressed by an active Trichophyton rubrum fungal infection. It may be assumed that the fungus has been weakened or deactivated by microwave treatment to such an extent that it cannot exert any signalling advantage or influence over the hosts innate immune system and has thus started to be successfully repelled. The progress duration being the natural growth-rate of the nail which varies with health, age, nutrition and other factors. The active (alive) state of the fungus can also be established by culturing samples to attempt to propagate the fungus in-vitro.

In summary this approach has detailed both in-vivo and in-vitro successful control of a fungal organism using a unique microwave energy treatment modality.

### REFERENCES

i Moyes D L, Shen C, Murciano C, Runglall M, Richardson J P, Arno M, Aldecoa-Otalora E, Naglik J R. 2014. Protection against epithelial damage during Candida albicans infection is mediated by PI3K/Akt and mammalian target of rapamycin signaling. J Infect Dis 209:1816-1826. http://dx.doi.org/10.1093/infdis/jit824.
ii Volling K, Thywissen A, Brakhage A A, Saluz H P. Phagocytosis of melanized Aspergillus conidia by macrophages exerts cytoprotective effects by sustained PI3K/Akt signalling. Cell Microbiol. 2011; 13(8):1130-1148. doi:10.1111fj.1462-5822.2011.01605.x
iii Santiago K, Bomfim G F, Criado P R, Almeida S R. Monocyte-derived dendritic cells from patients with dermatophytosis restrict the growth of Trichophyton rubrum and induce CD4-T cell activation. PLoS One. 2014; 9(11):e110879. Published 2014 Nov. 5. doi:10.1371/joumal.pone.0110879
iv Baltazar Lde M, Santos P C, Paula T P, et al. IFN-γ impairs Trichophyton rubrum proliferation in a murine model of dermatophytosis through the production of IL-1β and reactive oxygen species. Med Mycol. 2014; 52(3):293-302. doi:10.1093/mmy/myt011
v Campos M R, Russo M, Gomes E, Almeida S R. Stimulation, inhibition and death of macrophages infected with Trichophyton rubrum. Microbes Infect. 2006; 8(2):372-379. doi:10.1016/j.micinf.2005.07.028
vi Xu X, Liu T, Leng W, et al. Global gene expression profiles for the growth phases of Trichophyton rubrum. Sci China Life Sci. 2011; 54(7):675-682. doi:10.1007/s11427-011-4187-5
vii Martinez-Rossi N M, Jacob T R, Sanches P R, et al. Heat Shock Proteins in Dermatophytes: Current Advances and Perspectives. Curr Genomics. 2016; 17(2):99-111. doi:10.2174/1389202917666151116212437
viii Garcia-Madrid, L.A., Huizar-López, M. d. R., Flores-Romo, L. et al. Trichophyton rubrum manipulates the innate immune functions of human keratinocytes. cent.eur.j.biol. 6, 902-910 (2011). https-//doi.org/10.2478/si1535-011-0060-6
ix Komoto T T, Bitencourt T A, Silva G, Beleboni R O, Marins M, Fachin A L. Gene Expression Response of Trichophyton rubrum during Coculture on Keratinocytes Exposed to Antifungal Agents. Evid Based Complement Alternat Med. 2015; 2015:180535. doi:10.1155/2015/180535
x Whitney J L, Krockenberger M B, Day M J, Beatty J A, Dhand N K, Barrs V R. Immunohistochemical Analysis of Leucocyte Subsets in the Sinonasal Mucosa of Cats with Upper Respiratory Tract Aspergillosis. J Comp Pathol. 2016 August-October; 155(2-3):130-140. doi: 10.1016/j.jcpa.2016.08.001. Epub 2016 Aug. 27. PMID: 27576043.
xi Stephanie C. Casey, Virginie Baylot, Dean W. Felsher, M Y C: Master Regulator of Immune Privilege, Trends in Immunology, Volume 38, Issue 4, 2017, Pages 298-305, ISSN 1471-4906, https://doi.org/10.1016/j.it.2017.01.002.
xii Kahlfuss S, Kaufmann U, Concepcion A R, Noyer L, Raphael D, Vaeth M, Yang J, Pancholi P, Maus M, Muller J, Kozhaya L, Khodadadi-Jamayran A, Sun Z, Shaw P, Unutmaz D, Stathopulos P B, Feist C, Cameron S B, Turvey S E, Feske S. STIM1-mediated calcium influx controls antifungal immunity and the metabolic function of non-pathogenic Th17 cells. EMBO Mol Med. 2020 Aug. 7; 12(8):e11592. doi: 10.15252/emmm.201911592. Epub 2020 Jul. 1. PMID: 32609955; PMCID: PMC7411566.
xiii Rodaki, A., Young, T. and Brown, A. J. (2006) Effects of depleting the essential central metabolic enzyme fructose-1,6-bisphosphate aldolase on the growth and viability of Candida albicans: implications for antifungal drug target discovery. Eukaryot Cell. 5, 1371-1377
xiv Liu T, Xu X, Leng W, Xue Y, Dong J, Jin O. Analysis of gene expression changes in Trichophyton rubrum after skin interaction. J Med Microbiol. 2014 May; 63(Pt 5):642-648. doi: 10.1099/jmm.0.059386-0. Epub 2014 Feb. 28. PMID: 24586032; PMCID: PMC4042497.
xv Costa, M., Borges, C. L., Bailao, A. M., Meirelles, G. V., Mendonca, Y. A., Dantas, S. F., de Faria, F. P., Felipe, M. S., Molinari-Madlum, E. E., Mendes-Giannini, M. J.,
Fiuza, R. B., Martins, W. S., Pereira, M. and Soares, C. M. (2007) Transcriptome profiling of Paracoccidioides brasiliensis yeast-phase cells recovered from infected mice brings new insights into fungal response upon host interaction. Microbiology. 153, 4194-4207
xvi Tucey T M, Verma J, Harrison P F, Snelgrove S L, Lo T L, Scherer A K, Barugahare A A, Powell D R, Wheeler R T, Hickey M J, Beilharz T H, Naderer T, Traven A. Glucose Homeostasis Is Important for Immune Cell Viability during Candida Challenge and Host Survival of Systemic Fungal Infection. Cell Metab. 2018 May 1; 27(5):988-1006.e7. doi: 10.1016/j.cmet.2018.03.019. PMID: 29719235; PMCID: PMC6709535.
xvii Dongmei Shi, Jia Yang, Qiong Wang, Dongmei Li, Hailin Zheng, Huan Mei, Weida Liu, SOCS3 ablation enhances D C-derived Th17 immune response against Candida albicans by activating IL-6/STAT3 in vitro, Life Sciences, Volume 222, 2019, Pages 183-194, ISSN 0024-3205, doi: 10.1016/j.lfs.2019.03.009.
xviii Shuyuan Liu, Yinglong Hou, Weiguo Liu, Chunyan Lu, Weixin Wang, Shujuan Sun Components of the Calcium-Calcineurin Signaling Pathway in Fungal Cells and Their Potential as Antifungal Targets Eukaryotic Cell Apr. 2015, 14 (4) 324-334; DOI: 10.1128/EC.00271-14

1. A method of treating or preventing a fungal infection, said method comprising administering a subject suffering from, or predisposed to a fungal infection, an immunomodulatory amount or dose of microwave energy.

2. The method of claim 1, wherein the fungal infection is a fungal infection of a keratinised tissue or keratinised structure.

3. The method of claim 1, wherein the fungal infection is an onychomycosis condition of a nail.

4. The method of claim 1, wherein the fungal infection is caused or contributed to by any one or more of the below listed fungal pathogens:
(i) a dermatophyte;
(ii) a Fusarium;
(iii) a Microsporum sp.;
(iv) a Michrodochium sp.;
(v) a Epidermophyton sp.;
(vi) a Trichophyton sp.;
(vii) Trichophyton rubrum;
(viii) Trichophyton mentagrophytes; and
(ix) Epidermophyton floccosum.

5-7. (canceled)

8. The method of claim 1, wherein the subject is immunocompromised.

9. The method of claim 1, wherein the immunomodulatory dose does not kill or inactivate the fungal pathogen.

10-11. (canceled)

12. The method of claim 1, wherein the microwave energy is administered to modulate the activity, expression and/or function of the host innate and adaptive immune response and/or to modulate the expression, function and/or activity of those host immune factors, cytokines, chemokines, inflammatory factors, anti-inflammatory factors and/or apoptotic factors which are modulated by the fungal pathogen.

13-15. (canceled)

16. A method of downregulating one or more of:
(i) PI3K;
(ii) p-AKT;
(iii) IL-10;
(iv) EGR1;
(v) CD79B;
(vi) MYC; and/or
(vii) SOCS3;
in a subject, said method comprising administering the subject microwave energy.

17. A method of upregulating one or more of:
(i) IL-1β;
(ii) IL-6;
(iii) IL-2;
(iv) IL-12;
(v) (TNF)-α;
(vi) IFN-γ;
(vii) MHC class 2 APC;
(viii) CD80;
(ix) CD74;
(x) CD4;
(xi) T cell receptor;
(xii) NFkB;
(xiii) IL-18;
(xiv) MAPK1;
(xv) HSP; and
(xvi) MHC class 1.
in a subject, said method comprising administering the subject microwave energy.

18-22. (canceled)

23. The method of claim 1, wherein the microwave energy has a frequency of between about 500 MHz and about 200 GHz, or about 900 MHz and about 100 GHz, or about 900 MHz and about 100 GHz, or a frequency of 8 GHz.

24-26. (canceled)

27. The method of claim 1, wherein the microwave energy is administered as a single continuous dose or as a pulse dose comprising multiple, spaced apart individual doses.

28. The method of claim 1, wherein the microwave energy is administered at a power of 1 W, 2 W, 3 W, 4 W, about 5 W, 6 W, 7 W, 8 W, 9 W, 10 W, 11 W or 12 W.

29. The method of claim 1, wherein the microwave energy is administered for 1 second, 2 seconds, 3 seconds, 4 seconds, 5 seconds, 6 seconds, 7 seconds, 8 seconds, 9 seconds or 10 seconds.

30. The method of claim 27, wherein the multiple spaced apart doses are separated by a rest period of 1 second, 2 seconds, 3 seconds, 4 seconds, 5 seconds, 6 seconds, 7 seconds, 8 seconds, 9 seconds or 10 seconds.

31. (canceled)

32. The method of claim 27, wherein the microwave dose is repeated.

33. (canceled)

34. The method of claim 32, wherein each repeated dose is separated from another by a dose rest period.

35. (canceled)

36. The method claim 1, wherein the microwave energy is administered as a dose regime comprising three individual doses of 9W with five seconds rest between each individual dose.

37. The method of claim 36, wherein the dose regime is repeated or repeated five times over a period of 10 weeks.

38. (canceled)

39. The method of claim 1, wherein the microwave energy is administered as a dose regime comprising an initial dose of microwave energy designed to elevate the temperature of the area, tissue or structure being treated and a second dose designed to maintain a temperature in the area, tissue or structure being treated.

40. The method of claim 39, wherein the initial dose may comprise microwave energy at a power of 10 W, 15 W, 20 W or 25 W.

41. The method of claim 39, wherein the second dose may comprise microwave energy at a power of 1 W, 2 W, 3 W, 4 W, 5 W, 6 W, 7 W, 8 W, 9 W or 10 W.

42. The method of claim 39, wherein the first dose is administered for anywhere between 5 and 30 seconds.

43. The method of claim 39, wherein the second dose is administered for anywhere between 5 and 30 seconds.

44. The method of claim 39, wherein the first dose raises the temperature of the area, tissue or structure being treated to between about 30° C. and 60° C.

45. The method of claim 39, wherein the second dose raises the temperature of the area, tissue or structure being treated to between 20° C. and 60° C.

46. The method of claim 1, wherein the microwave energy is administered directly to the site of infection.

47. The method of claim 1, wherein the microwave energy is administered to the same or a different site.

48. The method of claim 1, wherein the microwave energy is administered to (i) the site of the fungal infection; (ii) a site proximal or adjacent to a fungal infection; or (iii) a site distal to the site of a fungal infection.

49. The method of claim 45, wherein the site to be treated is first hydrated or moistened.

50. The method of claim 49, wherein hydrating or moistening the site or sites to be treated comprises immersing the site or sites to be treated in a water bath to hydrate or moisten the site or sites.

51. The method of claim 49, wherein the site(s) to be treated is/are a fingernail(s) or toenail(s).

52. The method of claim 49, wherein the step of hydrating or moistening the site to be treated, includes or comprises the use of a hydration and/or lossy ionic fluid.

53. The method of claim 52, wherein the hydration and/or lossy ionic fluid buffers the energy closer to the surface of the site to be treated or within certain predetermined or defined regions of the site and/or its surrounding tissues and structures.

54. The method of claim 52, wherein the hydration and/or lossy ionic fluid comprises sodium chloride (NaCl).

55. The method of claim 48, wherein the site to be treated is pre-treated with Urea.

56. The method of claim 55, wherein the urea is applied to the site to be treated at a concentration of 10% to 50%.

57. The method of claim 55, wherein a urea pre-treatment is administered to the site to be treated before the site to be treated is hydrated or moistened.

58-67. (canceled)

68. The method of claim 1, wherein the microwave energy is administered to a or the keratinised structure using an apparatus comprising a microwave energy source and a microwave applicator formed, adapted and/or configured to deliver or administer microwave energy to the subject.

69. The method of claim 68, wherein the microwave energy, the applicator or the means of administering or delivering the microwave energy electrically matches the range of epsilon relative values of the keratinised structure to be treated.

70. The method of claim 68, wherein the applicator may be lubricated, coated or used with a buffer medium to remove airgap discontinuity between the applicator contacting and the keratinised structure to ensure good delivery of microwave energy.

71. The method of claim 70, wherein the buffer medium comprises propylene glycol, glycerine.

72. The method of claim 68, wherein the microwave energy source produces microwave energy at a single frequency and/or microwave energy across a range of frequencies.

73-85. (canceled)
